(19) United States
(12) Patent Application Publication
(10) Pub. No.: US 2008/0177076 A1
(43) Pub. Date: Jul. 24, 2008

(54) Processes for preparing substantially pure pantoprazole magnesium

(76) Inventors: Lilach Hedvanti, Doar Na Hefer (IL); Gideon Pilarski, Holon (IL)

Correspondence Address: KENYON & KENYON LLP, ONE BROADWAY, NEW YORK, NY, 10004, US

(21) Appl. No.: 11/982,888

(22) Filed: Nov. 5, 2007

Related U.S. Application Data
(60) Provisional application No. 60/857,420, filed on Nov. 6, 2006.

(51) Int. Cl. C07D 401/12

(52) U.S. Cl. 546/273.7

(57) ABSTRACT

Provided are processes for preparing substantially pure pantoprazole magnesium.

### CROSS-REFERENCE TO RELATED APPLICATIONS

This application claims the benefit of U.S. provisional application Ser. No. 60/857,420, filed Nov. 6, 2006, hereby incorporated by reference.

### FIELD OF THE INVENTION

The invention encompasses processes for preparing substantially pure pantoprazole magnesium.

### BACKGROUND OF THE INVENTION

Pantoprazole magnesium salt (“PNT-Mg”) has the chemical name 5-difluoromethoxy-2-[[3,4-dimethoxy-2-pyridinyl)methyl]sulfinyl]-1H-benzimidazole magnesium salt and the following chemical structure:

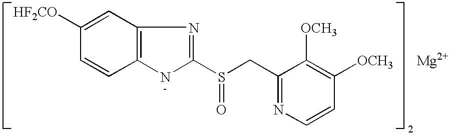

Pantoprazole and salts thereof are gastric acid secretion inhibitors, and are typically used as anti-ulcer agents. Pantoprazole is currently marketed by Altana under the trade name PANTOLOC® in the form of the sodium sesquihydrate salt.

U.S. Pat. No. 4,758,579 (“'579 patent”) to Byk Gulden discloses the compound pantoprazole, as well as its pharmacologically acceptable salts. '579 patent, col. 35, ll. 10-13. U.S. Pat. No. 6,124,464 (“'464 patent”) discloses a process for preparing pantoprazole magnesium by the reaction of pantoprazole with aqueous ammonia and magnesium sulfate in the presence of methanol. '464 patent, col. 9, ll. 45-61. The pantoprazole magnesium was reportedly recovered in 67% yield. Id.

U.S. Pat. No. 6,410,569 (“'569 patent”) and its continuation U.S. Pat. No. 6,686,379 (“'379 patent”) disclose three processes for preparing pantoprazole magnesium dihydrate. In the first process, pantoprazole magnesium dihydrate is prepared by the reaction of pantoprazole sodium sesquihydrate with magnesium dichloride hexahydrate in water. '569 patent, col. 2, l. 45 to col. 3, l. 7; '379 patent, col. 2, l. 45 to col. 3, l. 7. Using this process, the pantoprazole magnesium dihydrate was reportedly recovered in 90% yield. Id. In the second process, pantoprazole magnesium dihydrate is prepared by the reaction of pantoprazole sodium sesquihydrate with magnesium dichloride hexahydrate in a mixture of water and an organic solvent, such as isopropanol, ethanol, or acetone. '569 patent, col. 3, ll. 8-32; '379 patent, col. 3, ll. 8-32. In the third process, pantoprazole magnesium dihydrate is prepared by the reaction of pantoprazole with a basic magnesium salt, such as magnesium methylate, in the presence of 2-propanol. '569 patent, col. 3, l. 33 to col. 4, l. 5; '379 patent, col. 3, l. 33 to col. 4, l. 3.

Accordingly, there is a need in the art for an alternative process that can produce pantoprazole magnesium in high yield and purity, which employs reagents and conditions that are suitable for use on an industrial scale.

### SUMMARY OF THE INVENTION

The invention encompasses a process for preparing pantoprazole magnesium comprising: providing a mixture of pantoprazole acid, at least one magnesium base and at least one alcohol; and combining the mixture with at least one organic solvent to precipitate pantoprazole magnesium. Preferably, the pantoprazole magnesium thus prepared is substantially pure.

### DETAILED DESCRIPTION OF THE INVENTION

The invention encompasses substantially pure pantoprazole magnesium, as well as processes for preparing the substantially pure pantoprazole magnesium in high yield, using reagents and conditions that are suitable for use on an industrial scale.

As used herein, unless otherwise defined, the term “substantially pure pantoprazole magnesium” refers to pantoprazole magnesium salt having a purity of more than about 99% area by HPLC. Preferably, the substantially pure pantoprazole magnesium has a purity of more than about 99.5% area by HPLC, and more preferably more than about 99.9% area by HPLC.

As used herein, unless otherwise defined, the term “pantoprazole acid” refers to 5-difluoromethoxy-2-[[3,4-dimethoxy-2-pyridinyl)methyl]sulfinyl]-1H-benzimidazole having the following chemical structure:

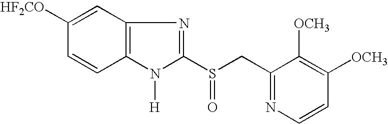

As used herein, unless otherwise defined, the term “room temperature” refers to a temperature of about 20° C. to about 25° C.

As used herein, unless otherwise defined, the term “organic solvent” refers to an organic solvent that contains less than about 0.5% by volume of water. Preferably, the organic solvent contains less than about 0.3% by volume of water, more preferably less than about 0.1% by volume of water, and most preferably is free of water.

The time periods described herein are time periods suitable for laboratory-scale preparations. One of ordinary skill in the art understands that suitable time periods will vary based upon the amounts of reagents present, and can adjust the time periods accordingly.

The invention encompasses a process for preparing pantoprazole magnesium comprising: providing a mixture of pantoprazole acid, at least one magnesium base and at least one alcohol; and combining the mixture with at least one organic solvent to precipitate pantoprazole magnesium.

One embodiment of the process is illustrated in Scheme 1 below.

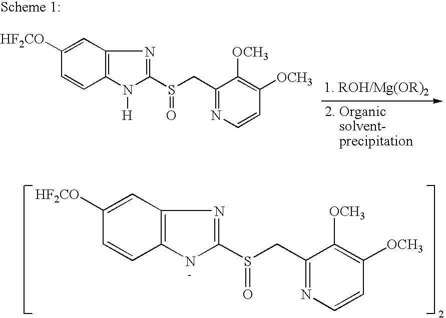

Typically, the alcohol is a linear or branched C₁-C₈ alcohol, such as methanol, ethanol, n-propanol, iso-propanol, n-butanol, iso-butanol, tert-butanol, n-pentanol, 2-pentanol, 3-pentanol, n-hexanol, 2-hexanol, 3-hexanol, n-heptanol, 1-heptanol, 2-heptanol, 3-heptanol, 4-heptanol, n-octanol, 2-octanol, 3-octanol, or 4-octanol. Preferably, the alcohol is methanol, ethanol, propanol, iso-propanol (“EPA”), butanol, or iso-butanol, and most preferably the alcohol is methanol.

Typically, the magnesium base is a magnesium alkoxide or alkyl magnesium. Preferably, the magnesium base is a magnesium alkoxide, and more preferably magnesium methoxide or magnesium ethoxide.

Typically, the organic solvent is a C₆-C₁₀ aromatic hydrocarbon, C₂-C₄ nitrile, C₃-C₈ ketone, or C₄-C₈ acetate. Preferably, the C₆-C₁₀ aromatic hydrocarbon is toluene. Preferably, the organic solvent is acetonitrile (“ACN”), acetone, or ethyl acetate. Most preferably, the organic solvent is toluene. The organic solvent typically acts as an anti-solvent that allows for the selective precipitation of the pantoprazole magnesium, while impurities, such as 5-difluoromethoxy-2-[[3,4-dimethoxy-2-pyridin-2-yl)methyl]sulfonyl]-1H-benzimide (“PNT-SO₂”), 5-difluoromethoxy-1H-Benzimidazole-2-thiol (“PNT-SH”), and 5-difluoromethoxy-2-[[3,4-dimethoxy-2-pyridinyl)methyl]thio]-1H-benzimidazole (“PFBS”) substantially remain in solution. The chemical structures of the PNT-SO₂, PNT-SH, and PFBS are as follows:

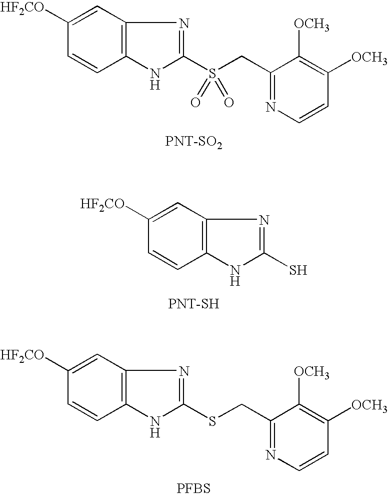

Typically, prior to the addition of the organic solvent, the mixture is heated at a temperature sufficient to facilitate the reaction between the pantoprazole acid and the magnesium base to obtain pantoprazole magnesium. Preferably, the mixture is heated to a temperature above about room temperature to about reflux temperature of the solvent, more preferably to a temperature of about 50° C. to about reflux temperature, and most preferably to about reflux temperature.

Typically, the mixture is maintained at the above temperature for a period of time sufficient to obtain the pantoprazole magnesium prior to the addition of the organic solvent. Preferably, the mixture is maintained for about 10 minutes to about 10 hours and more preferably for about 30 minutes to about 3 hours.

If the mixture is heated, the mixture may optionally be cooled prior to the addition of the organic solvent. Preferably, the mixture is cooled to a temperature of about 5° C. to about 40° C. and more preferably to about room temperature, prior to the addition of the organic solvent.

Preferably, after the addition of the organic solvent, the mixture is cooled to a temperature sufficient to precipitate the pantoprazole magnesium. Preferably, the mixture is cooled to a temperature of about 0° C. to about 25° C. and more preferably to a temperature of about 0° C. to about 5° C.

The precipitated pantoprazole magnesium may be recovered from the mixture by any method known to one of ordinary skill in the art. Preferably, the pantoprazole magnesium is recovered by collecting the precipitate by filtration, washing the precipitate, and drying the precipitate. Preferably, the precipitate is dried at a temperature of about 55° C., and preferably about 40° C. to about 75° C., under vacuum.

Typically, the pantoprazole magnesium is recovered in a yield of about 85% to about 98%, more preferably about 92% to about 97%, and most preferably about 97%.

Typically, the recovered pantoprazole magnesium is substantially pure. Preferably, the recovered pantoprazole magnesium has a purity of more than about 99.5% area by HPLC and more preferably a purity of more than about 99.9% area by HPLC. The remaining about 0.1% to about 1% of the material typically includes at least one of the PNT-SO₂, PNT-SH, or PNT-PFBS impurities described above.

Having described the invention with reference to certain preferred embodiments, other embodiments will become apparent to one skilled in the art from consideration of the specification. The invention is further defined by reference to the following examples describing in detail the processes for preparing the substantially pure pantoprazole magnesium. It will be apparent to those skilled in the art that many modifications, both to materials and methods, may be practiced without departing from the scope of the invention.

### EXAMPLES

### High Performance Liquid Chromatography (“HPLC”) Analysis:

Samples of pantoprazole magnesium prepared according to Examples 1 to 5 below were analyzed by HPLC using a Nova-Pak C18, 3.9×150 mm, 4 μm, model Wat. 086344 column equippend with an autosampler and an ultraviolet detector set at 285 nm. The column temperature was 30° C. and the autosampler temperature was 4° C. The flow rate was 1 ml/minute, the equilibration time was 10 minutes and the stop time was 35 minutes.

The sample volume was 20 μl and samples were diluted with a mixture of methanol and about 1.2% (w/w) ammonium hydroxide solution.

Samples were gradient eluted through the column with a mixture of eluent A (85% of a 0.01 M solution of di-ammonium hydrogen phosphate, adjusted to pH 7.5 with H₃PO₄; and 15% of a 7:3 mixture of acetonitrile:methanol) and eluent B (a 7:3 mixture of acetonitrile:methanol). The gradient was as follows:

| Time (min) | Eluent A (%) | Eluent B (%) |
|---|---|---|
| 0 | 86 | 14 |
| 10 | 86 | 14 |
| 35 | 42 | 58 |

The quantification limit of the above HPLC method is 0.05%.

### Example 1

### Preparation of Pantoprazole Mg

A flask (0.5 L) was loaded with methanol (150 ml), Mg(OEt)₂ (2.58 g) and pantoprazole acid (15 g). The resulting mixture was heated at reflux for 1 hour. The solvent was then evaporated to dryness. Methanol (15 ml) was added, the mixture was stirred, and toluene (135 ml) was added. The mixture was then heated to reflux and cooled to 2° C. over a period of 2 h. The precipitated solid was filtered, washed with toluene (20 ml) and dried at 55° C. under vacuum to yield pantoprazole magnesium (89.4% yield, Purity 99.9% area by HPLC).

### Example 2

### Preparation of Pantoprazole Mg

A flask (0.5 L) was loaded with methanol (150 ml), Mg(OEt)₂ (2.57 g) and pantoprazole acid (15 g). The resulting mixture was heated at reflux for 1 hour. The solvent was then evaporated to ˜1 volume. Toluene (135 ml) was added. The mixture was cooled to 2° C. over a period of 2 hours. The precipitated solid was then filtered, washed with toluene (20 ml) and dried at 55° C. under vacuum to yield pantoprazole magnesium (14.06 g, 88% yield, Purity 99.8% area by HPLC).

### Example 3

### Preparation of Pantoprazole Mg

A flask (0.25 L) was loaded with methanol (75 ml), Mg(OEt)₂ (1.29 g) and pantoprazole acid (7.5 g). The resulting mixture was heated at reflux for 10 min. Toluene (68 ml) was then added. The mixture was stirred for 15 hours at room temperature, then was cooled to 2° C. and stirred for an additional 2 hours. The precipitated solid was filtered, washed with toluene (15 ml) and dried at 55° C. under vacuum to yield pantoprazole magnesium (7.1 g, 92% yield, Purity 99.9% area by HPLC).

### Example 4

### Preparation of Pantoprazole Mg

A flask (0.25 L) was loaded with methanol (75 ml), Mg(OEt)₂ (1.29 g) and pantoprazole acid (7.5 g). The resulting mixture was heated at reflux for 15 min and then cooled to room temperature. Acetone (105 ml) was added. The mixture was heated to reflux and then cooled to 2° C. over a period of 2 h. The precipitated solid was filtered, washed with acetone (15 ml) and dried at 55° C. under vacuum to yield pantoprazole magnesium (7.15 g, 92% yield, Purity 99.9% area by HPLC).

### Example 5

### Preparation of Pantoprazole Mg

A flask (0.25 L) was loaded with methanol (75 ml), Mg(OEt)₂ (1.29 g) and pantoprazole acid (7.5 g). The resulting mixture was heated at reflux for 15 min and then cooled to room temperature. Acetonitrile (105 ml) was added. The mixture was then cooled to 2° C. over a period of 2 h. The precipitated solid was filtered, washed with acetone (15 ml) and dried at 55° C. under vacuum to yield pantoprazole magnesium (97% yield).

We claim:

1. A process for preparing pantoprazole magnesium comprising: providing a mixture of pantoprazole acid, at least one magnesium base and at least one alcohol; and combining the mixture with at least one organic solvent to precipitate pantoprazole magnesium.

2. The process of claim 1, wherein the precipitated pantoprazole magnesium has a purity of more than about 99 area percent as determined by HPLC.

3. The process of claim 1, wherein the precipitated pantoprazole magnesium has a purity of more than about 99.5 area percent as determined by HPLC.

4. The process of claim 1, wherein the precipitated pantoprazole magnesium has a purity of more than about 99.9 area percent as determined by HPLC.

5. The process of claim 1, wherein the alcohol is a linear or branched C₁-C₈ alcohol.

6. The process of claim 1, wherein the alcohol is methanol, ethanol, n-propanol, isopropanol, n-butanol, iso-butanol, tert-butanol, n-pentanol, 2-pentanol, 3-pentanol, n-hexanol, 2-hexanol, 3-hexanol, n-heptanol, 1-heptanol, 2-heptanol, 3-heptanol, 4-heptanol, n-octanol, 2-octanol, 3-octanol, or 4-octanol.

7. The process of claim 1, wherein the magnesium base is a magnesium alkoxide or alkyl magnesium.

8. The process of claim 1, wherein the magnesium base is a magnesium alkoxide.

9. The process of claim 8, wherein the magnesium alkoxide is magnesium methoxide or magnesium ethoxide.

10. The process of claim 1, wherein the mixture is heated prior to the addition of the organic solvent.

11. The process of claim 10, wherein the mixture is heated to a temperature of about 50° C. to about reflux temperature.

12. The process of claim 10, wherein the heated mixture is cooled prior to the addition of the organic solvent.

13. The process of claim 12, wherein the heated mixture is cooled to a temperature of about 5° C. to about 40° C.

14. The process of claim 1, wherein the mixture is maintained for a period of time sufficient to obtain pantoprazole magnesium prior to the addition of the organic solvent.

15. The process of claim 1, wherein the organic solvent is a C₆-C₁₀ aromatic hydrocarbon, a C₂-C₄ nitrile, a C₃-C₈ ketone, or a C₄-C₈ acetate.

16. The process of claim 1, wherein the organic solvent is toluene, acetonitrile, acetone, or ethyl acetate.

17. The process of claim 1, wherein, after the addition of the organic solvent, the mixture is cooled to a temperature sufficient to precipitate the pantoprazole magnesium.

18. The process of claim 17, wherein the mixture is cooled to a temperature of about 0C. to about 25° C.
